(19) United States
(12) Patent Application Publication
(10) Pub. No.: US 2023/0176017 A1
(43) Pub. Date: Jun. 8, 2023

(54) IN-SITU ANALYSIS OF METAL IONS IN VINYL ACETATE PRODUCTION

(71) Applicant: CELANESE INTERNATIONAL CORPORATION, Irving, TX (US)

(72) Inventors: Billy Charles TIPTON, Houston, TX (US); Laiyuan CHEN, Houston, TX (US); Yaw-Hwa LIU, Missouri City, TX (US); Dheeraj KUMAR, Pearland, TX (US)

(73) Assignee: CELANESE INTERNATIONAL CORPORATION, Irving, TX (US)

(21) Appl. No.: 17/997,018

(22) PCT Filed: Jun. 25, 2021
(86) PCT No.: PCT/US2021/039067

§ 371 Date: Oct. 24, 2022

Related U.S. Application Data
(60) Provisional application No. 63/045,371, filed on Jun. 29, 2020.

(51) Int. Cl. G01N 30/14; C07C 67/05; G01N 30/62

(52) U.S. Cl. CPC G01N 30/14; C07C 67/05; G01N 30/62

(57) ABSTRACT

Ion chromatography may be used for measuring the concentration of metal ions in various streams of a vinyl acetate production process. For example, a method may comprise: reacting ethylene, oxygen, and acetic acid in the presence of a catalyst and optionally a catalyst promotor like sodium acetate and/or potassium acetate to yield a crude vinyl acetate stream; measuring a concentration of a metal ion of the crude vinyl acetate stream and/or a stream downstream thereof with an ion chromatograph, wherein the metal ion is selected from the group consisting of Group I metal ions, Group II metal ions, transition metal ions, and any combination thereof.

### BACKGROUND

Vinyl acetate is produced by reacting ethylene, oxygen, and acetic acid in the presence of a catalyst (e.g., palladium, gold, and copper supported on a carrier). Further, the inclusion of compounds like sodium acetate and potassium acetate have been shown to increase the yield and selectivity of the reaction to vinyl acetate. Said sodium acetate and potassium acetate may be impregnated on the support and/or introduced with the feed to the reactor.

### BRIEF DESCRIPTION OF THE DRAWINGS

The following figures are included to illustrate certain aspects of the disclosure, and should not be viewed as exclusive configurations. The subject matter disclosed is capable of considerable modifications, alterations, combinations, and equivalents in form and function, as will occur to those skilled in the art and having the benefit of this disclosure.

FIG. 1 illustrates a process flow diagram of an example vinyl acetate production process of the present disclosure.

FIG. 2 is an ion chromatogram of a calibration standard and a vinyl acetate product.

### DETAILED DESCRIPTION

The present disclosure relates to methods and systems related to measuring the concentration of metal ion components in streams of the vinyl acetate production process. More specifically, the present systems and methods use ion chromatography for measuring said metal ion component concentrations. The ion chromatographs can be in-line with the vinyl acetate production system or off-line of the vinyl acetate production system. Preferably, the systems and methods use in-line, ion chromatography to allow for the measurements to occur more often and with less worker intervention.

In vinyl acetate production processes, metal ion components in the various streams can originate from a variety of locations. For example, Group I metals, (particularly sodium and potassium) are used in catalyst promoters to improve vinyl acetate yield and selectivity. Maintaining the proper concentration of the promoter across the reactor bed is critical to maintaining the “health” of the catalyst. In doing so, significant savings can be realized. There exists an optimal promoter concentration. Below this concentration there is an unfavorable shift in reactor products and high temperature areas (hot spots) may occur in the reactor bed packing. The catalyst can be rapidly damaged at high temperature due to sintering. This damage is irreparable, short of catalyst replacement.

Further, above the optimal promoter concentration, reaction rate and temperature drop considerably. During reactor operation, the promoter migrates from the entrance of the reactor toward the exit of the reactor. That is, the promoter is washed out of the reactor. Without promoter replenishment, promotor wash out would eventually lead to lower than optimal promoter concentration in the reactor, which can lead to premature catalyst aging or failure. Alternatively, the replenishment rate of the promoter can be too high. Generally, the addition of excessive promoter to the catalyst does not lead to irreparable damage if detected early, but loss of production rate and process efficiency is an undesirable outcome. Measuring the amount of promoter that leaves the reactor allows a mass balance to be performed. The rate of promoter addition (or replenishment) to the reactor is known. From these values, it is possible to determine if the promoter content of the reactor remains steady or is rising or falling.

Some vinyl acetate production processes include off-line analyses that use atomic absorption spectroscopy or atomic emission spectroscopy methods that require significant sample preparation (e.g., digestion of samples). Therefore, because of the required labor, these measurements are done infrequently (e.g., one to three times weekly). This does not allow for identifying spikes in sodium and/or potassium concentrations that would be indicative of an upset in the reactor. Advantageously, the methods and systems described herein allow for rapid monitoring of the sodium and/or potassium concentrations in crude vinyl acetate product or streams downstream thereof to identify and rectify reactor upsets and/or catalyst health rapidly.

Further, other metal ion components like iron, nickel, and/or chromium present in a stream of the vinyl acetate production process may indicate that corrosion is occurring in a system component. Monitoring the concentration of such metal ion components may provide operators with information regarding the rate of corrosion and unexpected increases in corrosion, which can be used to identify when to shut down the system or portion thereof for evaluation and/or maintenance. Further, corrosion products may also foul or poison the catalyst. Advantageously, the methods and systems described herein allow for monitoring of multiple metal components in a vinyl acetate stream, which further enhances the ability to monitor the system for needed adjustments to the processes and/or for identifying when other interventions (e.g., shut downs for evaluation and/or maintenance) need to occur.

Additionally, magnesium and calcium measurements can be useful in two ways. First, the presence of magnesium and calcium ion contaminants can be detected in the process. Secondly, magnesium and calcium ions can be included in specific standards used for calibration and performance checks of the ion chromatograph. The degradation of the analytical column(s) used in the ion chromatograph usually appears first in the chromatography of Group II metal ions of which magnesium and calcium are members. Worsening peak asymmetry or loss of resolution of these components can serve as an early warning that the analytical column(s) need maintenance or replacement.

Other instruments that are used for potassium measurements in a laboratory setting are atomic absorption (AA), inductively coupled plasma optical emission spectrometry (ICP-OES), and ICP-mass spectrometry (ICP-MS). These techniques require complex sample preparation that would be difficult to automate in vinyl acetate production systems. Further, said instruments have a relatively high cost of ownership and maintenance. The offline measurement of potassium ion has been attempted using an ion selective electrode (ISE). This met with limited success because the barrier membrane that provides for selective sensing is easily fouled or damaged by components found in the crude vinyl acetate product.

Advantageously, the methods and systems described herein use ion chromatography, which is relatively low cost, has simple methods of sample pre-treatment (degassing, dilution), has a low energy usage, and does not use high temperature flames, plasma, or strong radiofrequency fields.

While only some of the Group I metals, Group II metals, and transition metals are specifically described herein, other Group I metals, Group II metals, and transition metals may also be analyzed via ion chromatography to ascertain the concentration of said metal ions in the vinyl acetate streams at specific points in time or over a prolonged period.

Generally, the methods of the present disclosure include reacting ethylene, oxygen, and acetic acid in the presence of a catalyst and optionally a catalyst promotor like sodium acetate and/or potassium acetate to yield a crude vinyl acetate stream. The crude vinyl acetate stream and/or a stream downstream thereof may be analyzed with an ion chromatograph (in-line or off-line of the vinyl acetate production system) for the concentration of one or more metals. Preferably, between the crude vinyl acetate stream and/or a stream downstream thereof is condensed and cooled (e.g., to 80° C. or below) prior to the concentration measurement with the ion chromatograph. The temperature of the condensed phase can be reduced to lower its vapor pressure and reduce bubble formation in the components of the sample delivery system.

Examples of metal ions that may be analyzed by an ion chromatograph relative to a vinyl acetate production process include, but are not limited to, Group I metal ions, Group II ions, transition ions, and any combination thereof. More specific examples include, but are not limited to, sodium ions, potassium ions, magnesium ions, cesium ions, iron ions, nickel ions, chromium ions, and the like and any combination thereof.

The ion chromatograph may use columns containing an ion exchange media. This media is typically in the form of a resin. The ion exchange media interacts with ions to cause them to elute at different times. For the analyses described herein, resins suitable for use in the column preferably have low exchange capacity since the exchange capacity determines the eluent strength required to elute ions from the column. If the capacity is high, a concentrated eluent will be required. With increasing eluent concentration comes higher conductivity and thermal noise. Eluent conductivity and thermal noise should preferably be reduced as much as possible when using the non-suppressed technique. The analytical columns preferably include silica-based particles that can simultaneously analyze for monovalent and divalent cations. Analytical columns containing suitable resins are commercially available, which may include, but are not limited to, IC PAK™ Cation M/D (available from Waters Corporation), METROSEP™ C2 (available from Metrohm AG), YS-50 (available from Showa Denko), PRP-X200 (available from Hamilton Company), and the like.

In-line ion chromatographs may be located at any suitable distance from the sampling point. Preferably, this distance is 60 meters or less to minimize the time between sampling and analysis to avoid sampling delay. The sample after extraction from the vinyl acetate production process may be passed through a transfer system of tubes or pipes. While these tubes or pipes could be made of polymers, metal alloys are preferred for their strength and endurance in industrial applications. In general, the flow through the sample transfer system is adjusted so that the liquid entering from the process will arrive at the instrument in 60 seconds or less. Seamless metallic tubing is preferred over metallic pipe as tubing can be bent to produce wider radiused bends than pipe fittings and this reduces the pressure drop that occurs when a flowing liquid is forced to change direction. The use of tubing can also reduce the number of fittings required, thus reducing possible leakage sites. The use of poorly flushed components (e.g., settling or degassing vessels, filter bowls, tee or cross connections, and Bourdon gauges) within the sample transfer system should be avoided. This is particularly important when analyses at trace level are to be performed. When possible, the existing process pressure is used to drive the flow of the sample through the sample transfer system. If insufficient pressure is available, the internal diameter of the transfer tubing can be increased and/or a booster pump can be installed. To prevent the settling of particulate matter in the transfer tubing, the flowing liquid should have a linear velocity of about 1.0 meters per second (m/s) to about 3.0 m/s. Preferably, turbulent flow should be induced. With these criteria met, fresh sample can be delivered quickly and continuously to a location near the inlet of the in-line ion chromatograph. At this location, a self-cleaning “swirl” filter may be installed to reduce the particulate matter delivered to the in-line ion chromatograph. Only a small fraction of the liquid flowing in the transfer tubing passes through the filter and travels toward the inlet of the in-line ion chromatograph. The unused portion exiting the swirl filter is usually returned to the process through an adjustable metering valve. This sample transfer system is often called a “fast loop.” The filtered sample exiting the swirl filter may pass through a pressure reducing regulator and/or a metering valve depending on the inlet specifications of the in-line ion chromatograph. Without limitation, the sample presented at the inlet to the in-line ion chromatograph is preferably at a pressure of about 0.034 MPa to about 0.103 MPa (or approximately 5 psig to 15 psig), a temperature of about 25° C. to about 40° C., and a flow rate of about 10 mL/minute to about 50 mL/minute. One skilled in the art will recognize that a variety of system components can be placed between the vinyl acetate process stream and the ion chromatograph inlet to achieve such conditions.

For off-line ion chromatograph, the conditions of the sample may preferably be closer to ambient conditions to enhance worker safety.

When the sample enters the ion chromatograph, other sample conditioning steps may be performed. Examples include, but are not limited to, degassing, dilution, microfiltration, and the like, and any combination thereof. A volumetrically measured portion of the conditioned sample is introduced to the analytical column for ion speciation, ion detection, and analysis. The analytical column resides in a temperature-controlled oven. The analytical column can thus be maintained at a fixed temperature or a programmable thermal profile can be followed. Preferably, the fixed or programmable gradient is an about 20° C. gradient to an about 60° C. gradient. The eluent flow rate through the analytical column may be fixed or programmed to follow a flow profile. Preferably, the fixed or programmable flow profile is about 0.01 mL/minute to about 4 mL/minute. The concentration and/or composition of the eluent can be fixed or programmed to change. Preferably, the eluent concentration and composition are fixed (isocratic). In this case, the eluent is a mixture of water and nitric acid whose acid concentration is about 0.1 mM to about 20 mM. Ion detection can be achieved by a variety of detectors including, but not limited to, conductivity detectors, electrochemical detectors, UV/VIS detectors, and fluorescence detectors. Preferably, ion detection is achieved by a conductivity detector. One skilled in the art will recognize that an ion chromatograph can be configured and programmed in various ways to achieve the analysis goals.

The measurement of metal ion component concentration in the vinyl acetate stream of interest may occur in time intervals ranging from about 1 minute to about 6 hours (or about 1 minute to about 2 hours, or about 15 minutes to about 3 hours, or about 30 minutes to about 6 hours).

Further, the time intervals may vary. For example, if a metal ion component concentration is higher than expected, another measurement may be taken more quickly than planned (including immediately) to identify if the measured metal ion component concentration was an outlier or correct.

Further, the time intervals may vary based on the stage of the vinyl acetate production process. For example, in start-up, the metal ion component concentrate may be measured more frequently as compared to when the vinyl acetate reaction has reached a reasonably steady state.

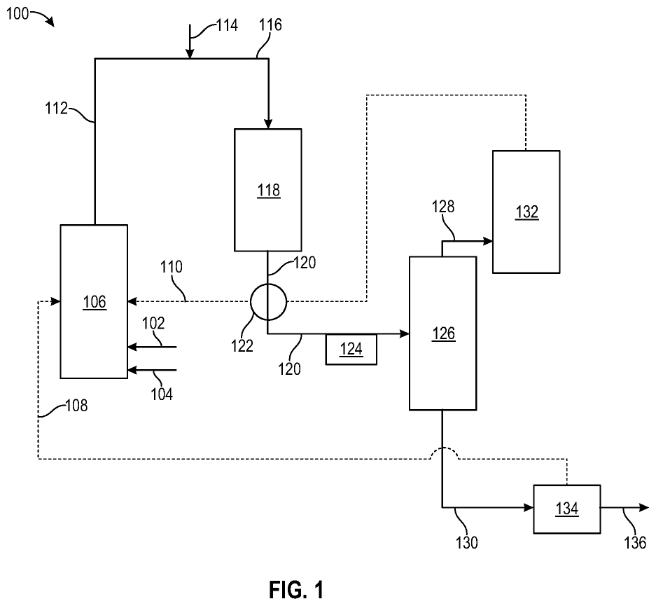

FIG. 1 illustrates a process flow diagram of an example vinyl acetate production process 100 of the present disclosure. Additional components and modifications may be made to the process 100 without changing the scope of the present invention. Further, as would be recognized by one skilled in the art, the description of the process 100 and related system uses streams to describe the fluids passing through various lines. For each stream, the related system has corresponding lines (e.g., pipes or other pathways through which the corresponding fluids or other materials may pass readily) and optionally valves, pumps, compressors, heat exchangers, or other equipment to ensure proper operation of the system whether explicitly described or not.

Further, the descriptor used for individual streams does not limit the composition of said streams to consisting of said descriptor. For example, an ethylene stream does not necessarily consist of only ethylene. Rather, the ethylene stream may comprise ethylene and a diluent gas (e.g., an inert gas). Alternatively, the ethylene stream may consist of only ethylene. Alternatively, the ethylene stream may comprise ethylene, another reactant, and optionally an inert component.

In the illustrated process 100, an acetic acid stream 102 and an ethylene stream 104 are introduced to a vaporizer 106. Optionally, ethane may also be added to the vaporizer 106. In addition, one or more recycle streams (illustrated as recycle streams 108 and 110) may also be introduced to the vaporizer 106. While the recycle streams 108 and 110 are illustrated as being directly introduced to the vaporizer 106, said recycle streams or other recycle streams may be combined (not shown) with the acetic acid stream 102 before introduction to the vaporizer 106.

The temperature and pressure of vaporizer 106 may vary over a wide range. The vaporizer 106 preferably operates at a temperature from 100° C. to 250° C., or from 100° C. to 200° C., or from 120° C. to 150° C. The operating pressure of the vaporizer 106 preferably is from 0.1 MPa to 2.03 MPa, or 0.25 MPa to 1.75 MPa, or 0.5 MPa to 1.5 MPa. The vaporizer 106 produces a vaporized feed stream 112. The vaporized feed stream 112 exits the vaporizer 106 and combines with an oxygen stream 114 to produce a combined feed stream 116 prior to being fed to a vinyl acetate reactor 118.

Regarding the general operating conditions of the vinyl acetate reactor 118, the molar ratio of ethylene to oxygen when producing vinyl acetate is preferably less than 20:1 in the vinyl acetate reactor 116 (e.g., 1:1 to 20:1, or 1:1 to 10:1, or 1.5:1 to 5:1, or 2:1 to 4:1). Further, the molar ratio of acetic acid to oxygen is preferably less than 10:1 in the vinyl acetate reactor 116 (e.g., 0.5:1 to 10:1, 0.5:1 to 5:1, or 0.5:1 to 3:1). The molar ratio of ethylene to acetic acid is preferably less than 10:1 in the vinyl acetate reactor 118 (e.g., 1:1 to 10:1, or 1:1 to 5:1, or 2:1 to 3:1). Accordingly, the combined feed stream 116 may comprise the ethylene, oxygen, and acetic acid in said molar ratios.

The vinyl acetate reactor 118 may be a shell and tube reactor that is capable, through a heat exchange medium, of absorbing heat generated by the exothermic reaction and controlling the temperature therein within a temperature range of 100° C. to 250° C., or 110° C. to 200° C., or 120° C. to 180° C. The pressure in the vinyl acetate reactor 118 may be maintained at 0.5 MPa to 2.5 MPa, or 0.5 MPa to 2 MPa.

Further, the vinyl acetate reactor 118 may be a fixed bed reactor or a fluidized bed reactor, preferably a fixed bed reactor that contains a catalyst suitable for acetoxylation of ethylene. Suitable catalysts for the production of vinyl acetate are described, for example, in U.S. Pat. Nos. 3,743,607; 3,775,342; 5,557,014; 5,990,344; 5,998,659; 6,022,823; 6,057,260; and 6,472,556, each of which is incorporated herein by reference. Suitable catalysts may comprise palladium, gold, vanadium, and mixtures thereof. Particular preference is given to the catalysts palladium acetate/potassium acetate/cadmium acetate and palladium acetate/barium acetolaurate/potassium acetate. In general, the palladium content of the catalyst is from 0.5 wt % to 5 wt %, or 0.5 wt % to 3 wt %, or 0.6 wt % to 2 wt %. When gold or one of its compounds is used, it is added in a proportion of 0.01 wt % to 4 wt %, or 0.2 wt % to 2 wt %, or 0.3 wt % to 1.5 wt %. The catalysts also preferably contain a refractory support, preferably a metal oxide such as silica, silica-alumina, titania, or zirconia, more preferably silica.

The vinyl acetate reaction in the reactor 118 produces a crude vinyl acetate stream 120. Depending on conversion and reaction conditions, the crude vinyl acetate stream 120 can comprise 5 wt % to 30 wt % vinyl acetate, 5 wt % to 40 wt % acetic acid, 0.1 wt % to 10 wt % water, 10 wt % to 80 wt % ethylene, 1 wt % to 40 wt % carbon dioxide, 0.1 wt % to 50 wt % alkanes (e.g., methane, ethane, or mixtures thereof), and 0.1 wt % to 15 wt % oxygen. Optionally, the crude vinyl acetate stream 120 may also comprise 0.01 wt % to 10 wt % ethyl acetate. The crude vinyl acetate stream 120 may comprise other compounds such as methyl acetate, acetaldehyde, acrolein, propane, and inerts such as nitrogen or argon. Generally, these other compounds, except for inerts, are present in very low amounts.

The crude vinyl acetate stream 120 passes through a heat exchanger 122 to reduce the temperature of the crude vinyl acetate stream 120. Preferably, the crude vinyl acetate stream 120 is cooled to a temperature of 80° C. to 145° C., or 90° C. to 135° C.

The systems and methods described herein measure the concentration of one or more metal ion components in the crude vinyl acetate stream 120 or a stream downstream thereof. As described above, the concentration of the metal ion components can be used to assess, among other things, the health of the system (e.g., the presence of corrosion), the health of the catalysts, the level of catalyst promotors and needed changes thereto, the health of the columns in the ion chromatograph, and any combination thereof.

As illustrated in FIG. 1, an ion chromatograph is in-line with the crude vinyl acetate stream 120. Here, a sample of the crude vinyl acetate stream 120 is collected and analyzed periodically by the in-line, ion chromatograph 124. Alternatively (not shown), the ion chromatograph may be offline from the system, and the methods may include collecting a sample from the crude vinyl acetate stream 120 and analyzing said sample in the offline, ion chromatograph.

The crude product exiting the reactor 118 is hot and is in the gas or vapor state. For timely analyses and dynamic control, samples should preferably be taken at the first instance that a representative liquid sample is available. Typically, this occurs a short distance downstream of the heat exchanger 122. If multiple heat exchangers are used, particularly in a parallel arrangement, the outlet streams of the condensers should be allowed to mix thoroughly before the sampling point. If a vessel is used to combine and mix condenser outlet streams, the residence time within that vessel should be maintained as short as possible so that a representative sample can be obtained.

Referring again to FIG. 1, the crude vinyl acetate stream 120 may then be conveyed to a separator 126 (e.g., a distillation column). Preferably, little to no condensation of the liquefiable components occurs and the cooled crude vinyl acetate stream 120 (post heat exchanger 122) is introduced to the separator 126 as gas. Accordingly, for proper operation of the in-line, ion chromatograph 124, components like a line or condensation vessel should be included to condense the crude vinyl acetate stream 120, so that a liquid is analyzed in the in-line, ion chromatograph 124. Similarly, if the ion chromatograph is off-line, components like a line or condensation vessel should be included to allow for safe handling of the sample by workers.

The energy to separate the components of the crude vinyl acetate stream 120 may be provided by the heat of reaction in the reactor 118. In some embodiments, there may be an optional reboiler (not illustrated) dedicated to increasing the separation energy within the separator 126.

The separator 126 separates the crude vinyl acetate stream 120 into at least two streams: an overheads stream 128 and a bottoms stream 130. The overheads stream 124 can comprise ethylene, carbon dioxide, water, alkanes (e.g., methane, ethane, propane or mixtures thereof), oxygen, and vinyl acetate. The bottoms stream 130 can comprise vinyl acetate, acetic acid, water, and potentially ethylene, carbon dioxide, and alkanes. Typically, metal ion components of interest in the present disclosure will condense into the bottoms stream 130. Therefore, an in-line, ion chromatograph (not illustrated) or an off-line, ion chromatograph (not illustrated) may be used in conjunction with the bottoms stream 130 for assessing the concentration of metal ion components therein. These ion chromatographs may be used in alternative of or in combination with ion chromatographs used with the crude vinyl acetate stream 120.

The overheads stream 128 may be further processed 132 (e.g., undergo further separations and/or be augmented with gases like ethylene and/or methane) to eventually produce the recycle stream 110. Again, the use of recycle stream 110 as a feed for the vaporizer 106 (either as is or previously mixed with another stream) is optional.

The bottoms stream 130 may be further processed 134 (e.g., undergo further purifications and separations) to eventually produce a vinyl acetate product stream 136 and the recycle stream 108. Again, the use of recycle stream 108 as a feed for the vaporizer 106 (either as-is or previously mixed with another stream) is optional.

A first nonlimiting example of the present disclosure is a method comprising: reacting ethylene, oxygen, and acetic acid in the presence of a catalyst and optionally a catalyst promotor like sodium acetate and/or potassium acetate to yield a crude vinyl acetate stream; and measuring a concentration of a metal ion of the crude vinyl acetate stream and/or a stream downstream thereof with an ion chromatograph, wherein the metal ion is selected from the group consisting of Group I metal ions, Group II metal ions, transition metal ions, and any combination thereof. The first nonlimiting example may further include one or more of: Element 1: wherein the ion chromatograph is in-line with a stream of the crude vinyl acetate stream and/or the stream downstream; Element 2: the method further comprising: condensing the crude vinyl acetate stream and/or the stream downstream before measuring the concentration of the metal ion; Element 3: wherein measuring continues over time at intervals ranging from about 1 minute to about 6 hours; Element 4: wherein the ion chromatograph uses a column comprising particles that can simultaneously analyze for monovalent and divalent cations; Element 5: wherein the ion chromatograph comprise a detector selected from the group consisting of a conductivity detector, an electrochemical detector, a UV/VIS detector, a fluorescence detector, and any combination thereof; and Element 6: wherein the metal ion is selected from the group consisting of sodium ion, potassium ion, magnesium ion, cesium ion, iron ion, nickel ion, and any combination thereof. Examples of combinations include, but are not limited to, Element 1 in combination with one or more of Elements 2-6; Element 2 in combination with one or more of Elements 3-6; Element 3 in combination with one or more of Elements 4-6; and two or more of Elements 4-6 in combination.

A second nonlimiting example embodiment is a system comprising: a reactor that contains a catalyst and that receives ethylene, oxygen, and acetic acid to produce a crude vinyl acetate stream; a heat exchanger that is downstream of the reactor; and an ion chromatograph that is in-line with the crude vinyl acetate stream, that is downstream of the heat exchanger, and that receives a sample from the crude vinyl acetate stream, wherein the ion chromatograph comprises a column capable of measure, in the crude vinyl acetate stream, a concentration of a metal ion selected from the group consisting of Group I metal ions, Group II metal ions, transition metal ions, and any combination thereof.

A third nonlimiting example embodiment is a system comprising: a reactor that contains a catalyst and that receives ethylene, oxygen, and acetic acid to produce a crude vinyl acetate stream; a heat exchanger that is downstream of the reactor; a separator that is downstream of the heat exchanger, that receives the crude vinyl acetate stream, and that separates the crude vinyl acetate stream into an overheads stream and a bottoms stream; and an ion chromatograph that is in-line with the bottoms stream and that receives a sample from the bottoms stream, wherein the ion chromatograph comprises a column capable of measure, in the bottoms stream, a concentration of a metal ion selected from the group consisting of Group I metal ions, Group II metal ions, transition metal ions, and any combination thereof.

The second and third nonlimiting example embodiments may further include one or more of: Element 7: wherein the ion chromatograph uses a column comprising particles that can simultaneously analyze for monovalent and divalent cations; Element 8: wherein the ion chromatograph comprise a detector selected from the group consisting of a conductivity detector, an electrochemical detector, a UV/VIS detector, a fluorescence detector, and any combination thereof; and Element 9: wherein the metal ion is selected from the group consisting of sodium ion, potassium ion, magnesium ion, cesium ion, iron ion, nickel ion, and any combination thereof.

Unless otherwise indicated, all numbers expressing quantities of ingredients, properties such as molecular weight, reaction conditions, and so forth used in the present specification and associated claims are to be understood as being modified in all instances by the term “about.” Accordingly, unless indicated to the contrary, the numerical parameters set forth in the following specification and attached claims are approximations that may vary depending upon the desired properties sought to be obtained by the incarnations of the present inventions. At the very least, and not as an attempt to limit the application of the doctrine of equivalents to the scope of the claim, each numerical parameter should at least be construed in light of the number of reported significant digits and by applying ordinary rounding techniques.

One or more illustrative incarnations incorporating one or more invention elements are presented herein. Not all features of a physical implementation are described or shown in this application for the sake of clarity. It is understood that in the development of a physical embodiment incorporating one or more elements of the present invention, numerous implementation-specific decisions must be made to achieve the developer's goals, such as compliance with system-related, business-related, government-related and other constraints, which vary by implementation and from time to time. While a developer's efforts might be time-consuming, such efforts would be, nevertheless, a routine undertaking for those of ordinary skill in the art and having benefit of this disclosure.

While compositions and methods are described herein in terms of “comprising” various components or steps, the compositions and methods can also “consist essentially of” or “consist of” the various components and steps.

To facilitate a better understanding of the embodiments of the present invention, the following examples of preferred or representative embodiments are given. In no way should the following examples be read to limit, or to define, the scope of the invention.

### EXAMPLES

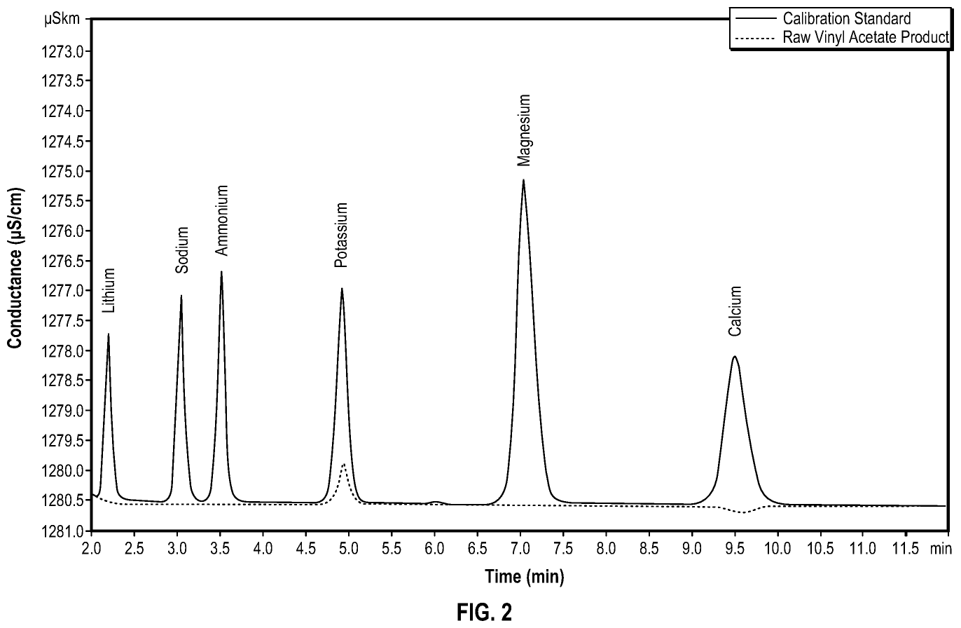

FIG. 2 is an ion chromatogram of a calibration standard and a vinyl acetate product. The samples were run in triplicate and the ion chromatograms overlap. Note the presence of potassium and sodium in the crude product sample. This illustrates that ion chromatography can be used to ascertain the concentration of various metal ions in streams of a vinyl acetate production process.

Therefore, the present invention is well adapted to attain the ends and advantages mentioned as well as those that are inherent therein. The particular examples and configurations disclosed above are illustrative only, as the present invention may be modified and practiced in different but equivalent manners apparent to those skilled in the art having the benefit of the teachings herein. Furthermore, no limitations are intended to the details of construction or design herein shown, other than as described in the claims below. It is therefore evident that the particular illustrative examples disclosed above may be altered, combined, or modified and all such variations are considered within the scope and spirit of the present invention. The invention illustratively disclosed herein suitably may be practiced in the absence of any element that is not specifically disclosed herein and/or any optional element disclosed herein. While compositions and methods are described in terms of “comprising,” “containing,” or “including” various components or steps, the compositions and methods can also “consist essentially of” or “consist of” the various components and steps. All numbers and ranges disclosed above may vary by some amount. Whenever a numerical range with a lower limit and an upper limit is disclosed, any number and any included range falling within the range is specifically disclosed. In particular, every range of values (of the form, “from about a to about b,” or, equivalently, “from approximately a to b,” or, equivalently, “from approximately a-b”) disclosed herein is to be understood to set forth every number and range encompassed within the broader range of values. Also, the terms in the claims have their plain, ordinary meaning unless otherwise explicitly and clearly defined by the patentee. Moreover, the indefinite articles “a” or “an,” as used in the claims, are defined herein to mean one or more than one of the element that it introduces.

1. A method comprising:
reacting ethylene, oxygen, and acetic acid in the presence of a catalyst and optionally a catalyst promotor like sodium acetate and/or potassium acetate to yield a crude vinyl acetate stream;
measuring a concentration of a metal ion of the crude vinyl acetate stream and/or a stream downstream thereof with an ion chromatograph, wherein the metal ion is selected from the group consisting of Group I metal ions, Group II metal ions, transition metal ions, and any combination thereof.

2. The method of claim 1, wherein the ion chromatograph is in-line with a stream of the crude vinyl acetate stream and/or the stream downstream.

3. The method of claim 1 further comprising:
condensing the crude vinyl acetate stream and/or the stream downstream before measuring the concentration of the metal ion.

4. The method of claim 1, wherein measuring continues over time at intervals ranging from about 1 minute to about 6 hours.

5. The method of claim 1, wherein the ion chromatograph uses a column comprising particles that can simultaneously analyze for monovalent and divalent cations.

6. The method of claim 1, wherein the ion chromatograph comprise a detector selected from the group consisting of a conductivity detector, an electrochemical detector, a UV/VIS detector, a fluorescence detector, and any combination thereof.

7. The method of claim 1, wherein the metal ion is selected from the group consisting of sodium ion, potassium ion, magnesium ion, cesium ion, iron ion, nickel ion, and any combination thereof.

8. A system comprising:
a reactor that contains a catalyst and that receives ethylene, oxygen, and acetic acid to produce a crude vinyl acetate stream;
a heat exchanger that is downstream of the reactor; and
an ion chromatograph that is in-line with the crude vinyl acetate stream, that is downstream of the heat exchanger, and that receives a sample from the crude vinyl acetate stream, wherein the ion chromatograph comprises a column capable of measure, in the crude vinyl acetate stream, a concentration of a metal ion selected from the group consisting of Group I metal ions, Group II metal ions, transition metal ions, and any combination thereof.

9. The system of claim 8, wherein the ion chromatograph uses a column comprising particles that can simultaneously analyze for monovalent and divalent cations.

10. The system of claim 8, wherein the ion chromatograph comprise a detector selected from the group consisting of a conductivity detector, an electrochemical detector, a UV/VIS detector, a fluorescence detector, and any combination thereof.

11. The system of claim 8, wherein the metal ion is selected from the group consisting of sodium ion, potassium ion, magnesium ion, cesium ion, iron ion, nickel ion, and any combination thereof.

12. A system comprising:
a reactor that contains a catalyst and that receives ethylene, oxygen, and acetic acid to produce a crude vinyl acetate stream;
a heat exchanger that is downstream of the reactor;
a separator that is downstream of the heat exchanger, that receives the crude vinyl acetate stream, and that separates the crude vinyl acetate stream into an overheads stream and a bottoms stream; and
an ion chromatograph that is in-line with the bottoms stream and that receives a sample from the bottoms stream, wherein the ion chromatograph comprises a column capable of measure, in the bottoms stream, a concentration of a metal ion selected from the group consisting of Group I metal ions, Group II metal ions, transition metal ions, and any combination thereof.

13. The system of claim 12, wherein the ion chromatograph uses a column comprising particles that can simultaneously analyze for monovalent and divalent cations.

14. The system of claim 12, wherein the ion chromatograph comprise a detector selected from the group consisting of a conductivity detector, an electrochemical detector, a UV/VIS detector, a fluorescence detector, and any combination thereof.

15. The system of claim 12, wherein the metal ion is selected from the group consisting of sodium ion, potassium ion, magnesium ion, cesium ion, iron ion, nickel ion, and any combination thereof.
